(19) United States
(12) Patent Application Publication
(10) Pub. No.: US 2022/0176000 A1
(43) Pub. Date: Jun. 9, 2022

(54) DEODORISING COMPOSITION

(71) Applicant: Ronald De Jesus Vargas Virviescas, Bogotá D.C. (CO)

(72) Inventor: Ronald De Jesus Vargas Virviescas, Bogotá D.C. (CO)

(73) Assignee: RV FARMACEUTICA LIMITADA, Bogotá (CO)

(21) Appl. No.: 17/600,582

(22) PCT Filed: Mar. 30, 2019
(86) PCT No.: PCT/IB2019/052646

§ 371 Date: Sep. 30, 2021

(51) Int. Cl. A61L 9/013

(52) U.S. Cl. CPC A61L 9/013

(57) ABSTRACT

The invention relates to liquid deodorizing compositions that comprise a mixture of alcohol, natural oils that include coconut oil, and excipient components, incorporated into an acceptable vehicle, its preparation process, methods to neutralize bad odors via the application of these compositions and their use. Deodorizing compositions can be applied by drip or spray.

### TECHNICAL FIELD

This invention falls within the field of chemistry, and specifically under deodorizing compositions used to eliminate unpleasant odors.

### OBJECT OF THE INVENTION

The invention relates to liquid deodorizing compositions that comprise a mixture of alcohol, natural oils and excipient components, incorporated into an acceptable vehicle. The compositions in this invention allow neutralizing or counteracting unpleasant odors, such as those produced by bacteria and excrement.

### STATE OF THE ART

Bad odors cause discomfort and affect people's well-being. Beyond simple rejection, unpleasant smells can cause irritation, nausea, dizziness and even intense headaches. The odors generated by bacteria in feces and urine are usually very unpleasant, diffuse easily into the environment and are very difficult to block or counteract.

Masking or dissipating bad odors present in an environment is an issue that cannot be solved completely by the application of air fresheners with scented compositions, as the specific olfactory quality of a bad smell combined with that of commonly known fragrances can result in a much more unpleasant sensation. Thus, it is only possible to mask bad odors using compositions specially designed or formulated with active agents, disinfectants, stabilizers, and fragrances evaporators with very specific properties. Odor masking compositions that include essences have been described. One of these is described in document CN103602534, which reveals a composition containing acetic acid, sodium sulfate, sodium hydrogen phosphate, coconut diethanolamide, 2,3-dihydroxysuccinic acid, and fragrances. The composition described therein is sprayed on the surface of a toilet and, after a slight rinsing, the bad smell is removed and cleaned.

U.S. patent Ser. No. 10/000,728 reveals water-based cleaning compositions preferably applied in aerosol form. Compositions include one or more organic molecules with hydrophilic groups as adhesion promoters; an anionic, non-ionic, cationic, amphoteric surfactant; and a propellant contained in a dispensing device.

The CN 103007320 patent application describes an odor removal agent comprising activated carbon blocks together with an aqueous liquid phase comprising honeysuckle extract, lemon extract, rose extract. This agent allows effectively removing odors from refrigerators.

Document WO 1997/00406 describes a cleaning agent for toilets, sinks, shower cabins and bathrooms, comprising cleaning additives in an aqueous solution, a surfactant, alkylamidobuteins, alkylbetains, fatty alcohol ethoxylates, and organic acids. U.S. Pat. No. 6,387,321 reveals a process for controlling toilet odors by using compositions comprising sodium hypochlorite and hydrogen peroxide.

Colombian patent CO2012031456 describes an environmental blocker that uses anaerobic agents in enclosed spaces and in a liquid composition that includes ethylene glycol monobutyl ether (Butyl Cellosolve®) as an active ingredient, and other agents such as alcohol, glycerin, pine oil, rose oil and eucalyptus oil. The composition described thereunder allows blocking the odors produced by excrement in toilets and washrooms.

Although the state of the art describes a wide variety of surface disinfectant and deodorizing compositions and agents, odor reduction strategies remain lacking, as there are limited options for eliminating these. Thus, a need exists to develop improved compositions that allow eliminating or neutralizing unpleasant odors.

### BRIEF DESCRIPTION OF THE INVENTION

The invention relates to a deodorizing composition that comprises a mixture of alcohol and natural oils as active ingredients together with one or more excipient components and incorporated into an acceptable vehicle. The invention also includes a process for manufacturing these compositions.

The compositions in the invention minimize the intensity of bad odors using active ingredients that do not possess, on their own, a significantly pleasant smell or fragrance. Additionally, the excipients included with the active ingredient enhance its deodorizing action for eliminating unpleasant odors.

### DETAILED DESCRIPTION

The deodorizing composition that is the subject of this invention comprises a mixture of alcohol and natural oils as active ingredients together with one or more excipient components and incorporated into an acceptable vehicle. The compositions under this invention can be manufactured using a process that involves mixing defined proportions of the components of the active ingredient and then adding each excipient component and incorporating them into an acceptable vehicle.

For the purposes of this invention, a deodorizing composition is defined as a composition that can neutralize or counteract an odor but does not mask it. Masking an odor means introducing a “masking” fragrance, which usually has a pleasant smell, and the intensity of the “masking” fragrance's smell modifies the unacceptable odor.

In turn, the terms “neutralize” and “counteract” indicate that the perceived intensity of the unacceptable odor is minimal or undetectable due to the presence of a neutralizing active ingredient, which may be present in concentrations that do not surpass the odor perception threshold and provide little or no odor, but which significantly reduces or eliminates the perception of the unpleasant odor. The deodorizing compositions under this invention provide both neutralize and counteract unpleasant odors in any application.

The active ingredient of the compositions under this invention is a mixture of established proportions of an alcohol and natural oils, one of which is coconut oil. The alcohol that makes up the active ingredient is selected from methanol, ethanol, isopropanol and mixtures thereof. In one form of the invention, the alcohol used for the active ingredient is ethanol. In another form of the invention, the alcohol used for the active ingredient is isopropanol. In another form of the invention, the alcohol used for the active ingredient is methanol. In another form of the invention, the active ingredient is an ethanol/methanol, methanol/isopropanol, or ethanol/isopropanol mix in equal or different ratios.

As far as the present invention is concerned, the term natural oil can be defined as an oleaginous substance of vegetable origin, rich in glycerol and fatty acids, liquid or solid at room temperature, which can be obtained from plant-based raw materials using known processes, such as distillation. The natural oils that make up the active ingredient are obtained from commonly known plants and are selected from coconut oil, cotton oil, palm oil, clove oil, bay leaf oil, lavender oil, jasmine oil, rose oil, pine oil, and eucalyptus oil, among others.

In one form of the invention, the natural oils used for the active ingredient are coconut oil and cotton oil in equal or different ratios. In another form of the invention, the natural oils used for the active ingredient are coconut oil and clove oil in equal or different ratios. In another form of the invention, the natural oils used for the active ingredient are coconut oil, clove oil, and cotton oil in equal or different ratios.

The ratio of alcohol to natural oils that make up the active ingredient can be 1:1; 1:2; 1:3; 2:1 or 3:1. In one form of the invention, the ratio of alcohol:natural oils used for the active ingredient is 1:1. In another form of the invention, the ratio of alcohol:natural oils used for the active ingredient is 1:2 or 2:1. In another form of the invention, the ratio of alcohol:natural oils used for the active ingredient is 1:3 or 3:1.

The active ingredient of the invention's compositions may be present in concentrations of between 5.0% and 95.0% (w/w) with respect to the overall composition, preferably between 20% and 70%, more preferably between 35.0% and 60.0%, and even more preferably between 40.0% and 55.0% (w/w) with respect to the overall composition. In one form of the invention, the active ingredient of the invention's composition is present in a concentration of 40.0% (w/w). In another form of the invention, the active ingredient of the invention's composition is present in a concentration of 48.0% (w/w) or 52.5% (w/w).

It will be evident to any person with knowledge of technical matters that concentrations of the active ingredient in the compositions under this invention may vary within these ranges and will depend on several factors including its intended use, the type of environment to be deodorized, and the type of excipient components and vehicle. Likewise, the active ingredient of the compositions under this invention may behave either as substance evaporator, disinfectant, bactericide, aromatic substance, or as an agent to form thin films on surfaces.

The active ingredient used in this invention, can be adapted, together with excipients and an acceptable vehicle, to obtain deodorizing compositions that can be used to neutralize or counteract bad odors. Excipients may include one or more surfactants, wetting agents, emollients, antioxidants, sequestrants, dispersants, dyes, pH regulators, and aromatic agents.

Excipient components of the deodorizing compositions under this invention are known and commonly employed by techniques for liquid compositions and are present in concentrations of between 10.0% and 90.0% (w/w) with respect to the overall composition, preferably between 30.0% and 70.0%, more preferably between 40.0% and 60.0%, and even more preferably between 45.0% and 55.0% (w/w) with respect to the overall composition. In one form of the invention, excipient components are present at concentrations between 35.0% and 65.0% (w/w).

Excipient components may include essences and/or fragrances that provide the compositions of the invention with a pleasant aroma. The essences and/or fragrances of these compositions could potentially act in association with the active ingredient but are not necessary for the active ingredient to exert its odor-neutralizing action. The essences and fragrances are those known and commonly used by techniques for creating liquid compositions, and may include floral essences, vanilla essence, mint essence, green apple essence, among others.

The excipient components of the compositions under this invention may include, among others, bleaching, bactericidal and disinfectant agents commonly known and used by the technique, including vinegar, apple cider vinegar, dicalcium phosphate, sodium diacetate, sodium bicarbonate and methylene blue. Additionally, the excipients of the compositions of this invention may include sequestering agents known and commonly used by the technical field, including EDTA.

For the purposes of this invention, the term acceptable vehicle refers to diluents such as purified water, alcohols, polyols, glycerin and mixtures thereof, which allow obtaining liquid compositions, either as solutions, emulsions, suspensions or dispersions. The alcohols and polyols that used in the acceptable vehicle of the compositions of this invention are those known and commonly employed in the technical field. In one form of the invention, the acceptable vehicle is a mixture of water and glycerin or a water:polyol mixture. In another form of the invention, the acceptable vehicle is purified water.

In one form of the invention, the deodorizing compositions are as follows:

TABLE 1

| Component | | Range (% w/w) |
|---|---|---|
| Active Ingredient | Alcohol | 5.0-50.0 |
| | Natural Oils | 1.0-40.0 |

| | |
|---|---|
| Excipient components | 1.0-90.0 |

In an other form of the invention, the deodorizing compositions are as follows:

TABLE 2

| Component | | Range (% w/w) |
|---|---|---|
| Active Ingredient | Ethanol | 10.0-40.0 |
| | Natural Oils | 3.0-30.0 |
| Excipient components | Apple cider vinegar | 1.0-10.0 |
| | EDTA | 0.1-10.0 |
| | Methylene blue | 0.1-10.0 |
| | Sodium bicarbonate | 0.1-10.0 |
| | Vanilla essence | 0.1-15.0 |
| | Mint essence | 0.1-15.0 |
| | Apple essence | 0.1-15.0 |
| | Lavender essence | 0.1-15.0 |
| | Purified water | 5.0-95.0 |

The deodorizing compositions of this invention can be packaged in containers suitable for direct, drip or spray application using dispensers known in the technical field.

The invention also provides a method to neutralize or counteract bad odors on an object or in an environment, which requires putting an adequate amount of the deodorizing compositions of this invention in contact with the object or environment to be deodorized. In one form of the invention, the object to be deodorized with the compositions of this invention is a toilet, pipe, or washroom, and the environment is a closed environment where unpleasant odors are present.

Deodorizing compositions under this invention can be used in a wide variety of objects or environments where it is necessary to neutralize or counteract bad odors. As such, this invention also contemplates the use of deodorizing compositions to neutralize or counteract unpleasant odors from various sources, such as odors produced by pollution, by decomposing organic matter, or by aerobic or anaerobic bacteria.

In one form of the invention, the deodorizing compositions of the invention are applied directly to the water contained in a pipe or a toilet to neutralize the bad odors generated there. In one form of the invention, the deodorizing compositions can be used to neutralize the odors produced by animal or human excrement in a toilet, a washroom or a basin, for which an adequate amount of the deodorizing composition is applied before or during the disposal of excrement.

The following examples illustrate the invention, but in no way do they restrict its concept.

### Examples

### Example 1. Preparation of a Deodorizing Composition

7.0 ml of coconut oil, 3.0 ml of clove oil and 14.0 ml of cotton oil were placed in container A. Then 9.0 m of vanilla fragrance; 4.0 ml of mint fragrance; 8.0 ml of green apple fragrance and 4.0 ml of lavender fragrance was added.

In container B, 2.0 grams of EDTA were dissolved in 9.5 ml of purified water and 1.5 grams of methylene blue, 5.0 grams of sodium bicarbonate, 9.0 ml of apple cider vinegar, and 24.0 ml of ethanol were added.

Finally, all the contents of container B were incorporated into container A to obtain 100 mL of deodorizing composition with the percentages (w/w) indicated in Table 3:

TABLE 3

| Component | % (w/w) |
|---|---|
| Ethanol | 24.0 |
| EDTA | 2.0 |
| Coconut oil | 7.0 |
| Vanilla essence | 9.0 |
| Mint essence | 4.0 |
| Green apple essence | 8.0 |
| Clove oil | 3.0 |
| Apple cider vinegar | 9.0 |
| Sodium bicarbonate | 5.0 |
| Lavender essence | 4.0 |
| Cotton oil | 14.0 |
| Methylene blue | 1.5 |
| Purified water q.s. | 100.0 |

### Example 2. Deodorizing Compositions

Following the same process indicated in Example 1, the appropriate quantities were arranged to prepare 100 mL of three deodorizing compositions (T, W, Z), with the percentages (w/w) indicated in Table 4.

TABLE 4

| | % (w/w) | | |
|---|---|---|---|
| Component | T | W | Z |
| Ethanol | 20.0 | 23.0 | 30.0 |
| EDTA | 1.0 | 1.5 | 1.8 |
| Coconut oil | 5.0 | 9.0 | 6.0 |
| Vanilla essence | 8.0 | 6.0 | 7.0 |
| Mint essence | 3.0 | 3.0 | 5.0 |
| Green apple essence | 7.0 | 9.0 | 7.0 |
| Clove oil | 2.0 | 2.5 | 3.5 |
| Apple cider vinegar | 7.0 | 6.0 | 8.0 |
| Sodium bicarbonate | 3.0 | 4.0 | 6.0 |
| Lavender essence | 3.0 | 3.0 | 5.0 |
| Cotton oil | 13.0 | 18.0 | 12.0 |
| Methylene blue | 1.0 | 2.0 | 1.7 |
| Purified water q.s. | 100.0 | 100.0 | 100.0 |

### Example 3. Sensory Analysis Test

Five trained evaluators performed a sensory analysis unpleasant odor test in an enclosed environment (unventilated bathroom) 2, 5, 10 and 15 minutes after excrement had been disposed of in a conventional toilet, to which 0.5 milliliters of the deodorizing composition in Example 1 had previously been added.

The trained evaluators entered the closed environment, perceived the odor to determine its intensity, and assigned it a score according to the scale in Table 5. The results obtained during the test can be found in Table 6.

TABLE 5
Odor scoring scale used by evaluators for the sensory analysis test

| Score | Odor Description |
|---|---|
| 0 | Bad odor not present |
| 10 | Barely noticeable bad odor |
| 20 | Slight bad odor |
| 25 | Moderately bad odor |
| 50 | Strong bad odor |
| 100 | Extremely strong bad odor |

TABLE 6
Sensory analysis test results

| | | | | | | | Standard |
|---|---|---|---|---|---|---|---|
| Evaluator | A | B | C | D | E | Average | deviation |
| Score at | 25 | 25 | 20 | 10 | 20 | 20 | 6.1 |
| the four | 20 | 10 | 10 | 10 | 10 | 12 | 4.5 |
| evaluated | 0 | 0 | 10 | 0 | 0 | 2 | 4.5 |
| moments | 0 | 0 | 0 | 0 | 0 | 0 | 0 |

According to the test results, none of the trained evaluators perceived a strong or extremely strong bad smell after 2 minutes, so the deodorizing composition tested managed to neutralize the unpleasant odor in a short time.

1) A deodorizing composition comprising a mixture of alcohol and natural oils as active ingredients, where one of the natural oils is coconut oil, together with one or more excipient components and incorporated into an acceptable vehicle.

2) The alcohol that makes up the active ingredient of the deodorizing composition under claim 1 is selected from methanol, ethanol, isopropanol and mixtures thereof.

3) The alcohol that makes up the active ingredient of the deodorizing composition under claim 1 is ethanol.

4) The natural oils that make up the active ingredient of the deodorizing composition under claim 1 are selected from cotton oil, clove oil, mint oil, laurel oil, and mixtures thereof.

5) The natural oils that make up the active ingredient of the deodorizing composition under claim 1 are selected from coconut oil, cotton oil, and clove oil.

6) The deodorizing composition under claim 1 is characterized by an alcohol:natural oils ratio of 1:1; 1:2; 1:3; 2:1 or 3:1.

7) The active ingredient of the deodorizing composition under claim 1 is a mixture of (ethanol):(coconut oil, cotton oil and clove oil) in a 1:1 ratio.

8) The deodorizing composition under claim 1 is characterized because its active ingredient is present in concentrations that range from 35.0% to 55.0% (w/w) of the overall composition.

9) The excipient components of the deodorizing composition under claim 1 are selected from aromatic agents, chelating agents, essences, pH stabilizers and dyes.

10) The deodorizing composition under claim 1 is characterized by the following composition: ethanol 10.0% to 40.0%; natural oils 3.0% to 30.0%; apple cider vinegar 1.0% to 10.0%; EDTA 0.1% to 10.0%; methylene blue 0.1% to 10%; sodium bicarbonate 0.1% to 10.0%; vanilla essence 0.1% to 15.0%; mint essence 0.1% to 15.0%; green apple essence 0.1% to 15.0%; lavender essence 0.1% to 15.0%; purified water q.s. 100.0%.

11) The deodorizing composition under claim 1 is characterized by the following composition (% w/w):ethanol 20.0%; coconut oil 5.0% to 2.0%; cotton oil 13.0%; clove oil 2.0%; apple cider vinegar 7.0%; EDTA 1.0%; methylene blue 1.0%; sodium bicarbonate 3.0%; vanilla essence 8.0%; mint essence 3.0%; green apple essence 7.0%; lavender essence 3.0%; purified water q.s. 100.0%.

12) The deodorizing composition under claim 1 is characterized by the following composition (% w/w):ethanol 24.0%; coconut oil 7.0%; cotton oil 14.0%; clove oil 3.0%; apple cider vinegar 7.0%; EDTA 2.0%; methylene blue 1.5%; sodium bicarbonate 5.0%; vanilla essence 9.0%; mint essence 4.0%; green apple essence 8.0%; lavender essence 4.0%; purified water q.s. 100.0%.

13) The deodorizing composition under claim 1 is characterized by the following composition (% w/w):ethanol 30.0%; coconut oil 6.0%; cotton oil 12.0%; clove oil 3.5%; apple cider vinegar 8.0%; EDTA 1.8%; methylene blue 1.7%; sodium bicarbonate 6.0%; vanilla essence 7.0%; mint essence 5.0%; green apple essence 7.0%; lavender essence 4.0%; purified water q.s. 100.0%.

14) A method to neutralize or counteract bad odors on an object or in an environment, including the step of putting an adequate amount of the deodorizing composition in accordance with claim 1 in contact with the object or environment to be deodorized.

15) A method of neutralizing or masking unpleasant odors comprising the step of applying a deodorizing composition according to claim 1.
